(19) United States
(12) Patent Application Publication
(10) Pub. No.: US 2018/0187248 A1
(43) Pub. Date: Jul. 5, 2018

(54) GENE SEQUENCING CHIP, GENE SEQUENCING APPARATUS AND GENE SEQUENCING METHOD

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Fengchun PANG, Beijing (CN); Peizhi CAI, Beijing (CN); Yue GENG, Beijing (CN)

(21) Appl. No.: 15/720,159

(22) Filed: Sep. 29, 2017

(30) Foreign Application Priority Data
Jan. 3, 2017 (CN) 201710002779.4

(51) Int. Cl. C12Q 1/68; B01L 3/00

(52) U.S. Cl. CPC C12Q 1/6837; C12Q 1/6869; C12Q 2565/513; C12Q 1/6806; B01L 3/5027

(57) ABSTRACT

The present disclosure provides a gene sequencing chip, a gene sequencing apparatus and a gene sequencing method. The gene sequencing chip comprising: a transparent first substrate; a second substrate disposed opposite to the first substrate; a first electrode disposed on the first substrate, which is a transparent electrode; an electronic ink layer disposed between the first substrate and the second substrate; and a microporous layer disposed on a side of the second substrate away from the first substrate. Micropores is formed at a position in the microporous layer corresponding to the first electrode.

### CROSS-REFERENCE TO RELATED APPLICATION

This application claims priority to Chinese Patent Application No. 201710002779A, filed on Jan. 3, 2017 in the Chinese Intellectual Property Office, the disclosure of which is incorporated herein by reference.

### FIELD OF THE INVENTION

The present disclosure relates to the field of gene sequencing, and more specifically relates to a gene sequencing chip, a gene sequencing apparatus and a gene sequencing method.

### BACKGROUND OF THE INVENTION

Gene sequencing technology is the most common technology in modern molecular biology research. Developed from the first generation of gene sequencing technology in 1977, gene sequencing technology has been made considerable development, comprising the first generation of sanger's sequencing technology, the second generation of high-throughput sequencing technology, the third generation of single molecule sequencing technology and the fourth generation of nanopore sequencing technology. However the main sequencing technology in current market is still based on the second generation of high-throughput sequencing.

The second generation of high-throughput sequencing technology mainly comprises Illumina sequencing (sequencing by synthesis), Thermo Fisher's ion semiconductor sequencing and sequencing by ligation, and pyrophosphate sequencing for Roche.

Fluorescence labeling is required in the method of sequencing by synthesis for Illumina and sequencing by ligation for Thermo Fisher, and laser light source and optical systems are also need to provided. Roche's pyrophosphate sequencing has no laser light source and optical systems, but fluorescent labeling is required. An ion sensor and two field-effect transistors are manufactured by CMOS process in the ion semiconductor sequencing.

Because fluorescence labeling is required in the method of sequencing by synthesis for Illumina and sequencing by ligation for Thermo Fisher, and laser light source and optical systems are also need to provided, such that the sequencing is more complicated and the sequencing time and cost is increased. The ion-semiconductor sequencing method due to the use of CMOS process to produce an ion sensor and two field-effect transistors which are difficult to manufacture, is therefore difficult to be achieved.

### SUMMARY OF THE INVENTION

In order to solve the above-mentioned problems of the prior art, the present disclosure provides a gene sequencing chip which does not require a laser light source and an optical system as well as any field-effect transistor. Therefore the manufacturing process is simple and can greatly reduce the manufacturing difficulty and cost. The present disclosure also relates to a gene sequencing apparatus comprising the gene sequencing chip.

In addition, the present disclosure also provides a gene sequencing method by which the gene sequencing apparatus of the present disclosure is used. Gene sequencing method in the present disclosure is capable of performing gene sequencing conveniently and simply without fluorescent labeling for deoxyribonucleotides.

The present invention provides a gene sequencing chip comprising: a transparent first substrate; a second substrate disposed opposite to the first substrate; a first electrode disposed on the first substrate, which is a transparent electrode; an electronic ink layer disposed between the first substrate and the second substrate; and a microporous layer disposed on a side of the second substrate away from the first substrate. Micropores are formed at a position in the microporous layer corresponding to the first electrode. The electronic ink layer comprises a plurality of microcapsules, each of which comprises positively charged white particles and negatively charged black particles.

According to an embodiment of the present disclosure, an ion-sensitive film is disposed on a side of the micropores close to the second substrate.

According to an embodiment of the present disclosure, the ion-sensitive film is made of silicon nitride.

According to an embodiment of the present disclosure, the gene sequencing chip further comprises a second electrode which is disposed on the second substrate. According to an embodiment of the present disclosure, the first electrode is disposed on a side of the first substrate close to the second substrate, and the second electrode is disposed on a side of the second substrate close to the first substrate. According to an embodiment of the present disclosure, both the first electrode and the second electrode are block electrodes, and projections of the first electrode and the second electrode on the first substrate are overlapped with each other.

According to an embodiment of the present disclosure, projections of the first electrode, the second electrode, and the micropores on the first substrate are overlapped with each other.

According to an embodiment of the present disclosure, the first electrode is a planar electrode bespreading the first substrate, and the second electrode is a block electrode.

Alternatively, the second electrode is a planar electrode bespreading the second substrate, and the first electrode is a block electrode.

According to an embodiment of the present disclosure, a first signal wire transmitting a voltage to the first electrode is disposed on the first substrate, and a second signal wire transmitting a voltage to the second electrode is disposed on the second substrate.

According to an embodiment of the present disclosure, the first electrode is made of indium tin oxide (ITO), and the second electrode, the first signal wire, the second signal wire is made of ITO, molybdenum, Aluminum, copper and the like, and the microporous layer is made of silicon nitride or silicon oxide.

The present disclosure further provides a gene sequencing chip comprising: a transparent first substrate; a second substrate disposed opposite to the first substrate; a second electrode disposed on the second substrate; an electronic ink layer disposed between the first substrate and the second substrate; and a microporous layer disposed on a side of the second substrate away from the first substrate. Micropores are formed at a position in the microporous layer corresponding to the first electrode.

The present disclosure also provides a gene sequencing apparatus comprising the gene sequencing chip according to the present disclosure.

According to an embodiment of the present disclosure, the gene sequencing apparatus further comprises an image acquisition device which is disposed on a side of the first substrate away from the second substrate and is configured to capture the color change of a part of the electronic ink layer close to the first substrate.

The present disclosure also provides a gene sequencing method comprising the following steps:

DNA microspheres containing DNA strands are added to the micropores of the gene sequencing chip for PCR amplification;

a voltage is applied to the first electrode such that an electric field is formed between the first substrate and the second substrate, the direction of which is directed from the first substrate to the second substrate;

four types of deoxyribonucleoside triphosphates are added to the micropores successively and detecting whether or not the color of a part of the electronic ink layer close to the first substrate is changed; and

The type of basic group on the DNA strand is determined according to the fact that which type of the deoxyribonucleoside triphosphate is added when the color of a part of the electronic ink layer close to the first substrate is changed. According to an embodiment of the present disclosure, the deoxyribonucleoside triphosphate is a reversible termination of deoxyribonucleoside triphosphate. The gene sequencing method further comprises washing the reversible termination of deoxyribonucleoside triphosphate added into the micropores and adding mercapto-reagent.

According to an embodiment of the present disclosure, an image acquisition device is disposed on a side of the first substrate away from the second substrate and is configured to capture the color change of a part of the electronic ink layer close to the first substrate.

The present disclosure also provides a gene sequencing method comprising the following steps:

DNA microspheres containing DNA strands are added to the micropores of the gene sequencing chip for PCR amplification;

a voltage is applied to the second electrode such that an electric field is formed between the first substrate and the second substrate, the direction of which is directed from the first substrate to the second substrate;

four types of deoxyribonucleoside triphosphates are added to the micropores successively and detecting whether or not the color of a part of the electronic ink layer close to the first substrate is changed; and

The type of basic group on the DNA strand is determined according to the fact that which type of the deoxyribonucleoside triphosphate is added when the color of a part of the electronic ink layer close to the first substrate is changed. According to an embodiment of the present disclosure, the deoxyribonucleoside triphosphate is a reversible termination of deoxyribonucleoside triphosphate. The gene sequencing method further comprises washing the reversible termination of deoxyribonucleoside triphosphate added into the micropores and adding mercapto-reagent.

### BRIEF DESCRIPTION OF THE DRAWINGS

The accompanying drawings constituting a part of the specification are intended to provide a further understanding of the present disclosure and explain the disclosure together with the following preferred embodiments, but should not be considered as limiting the scope of the disclosure. In the drawings:

FIG. 1 shows a cross-sectional view taken along the line A-A′ in FIG. 2 and FIG. 3 of a gene sequencing chip according to the present disclosure;

FIG. 2 shows a plan view of a first substrate of a gene sequencing chip in FIG. 1;

FIG. 3 shows a plan view of a second substrate of the gene sequencing chip in FIG. 1;

FIG. 4 shows a cross-sectional view of the gene sequencing chip of FIG. 1 in which complementary basic groups pairing reaction occurs;

FIG. 5-1 shows the color of a part of the electronic ink layer of the gene sequencing chip in FIG. 1 close to the first substrate when complementary basic groups pairing reaction has not occurred;

FIG. 5-2 shows the color of a part of the electronic ink layer of the gene sequencing chip in FIG. 1 close to the first substrate when complementary basic groups pairing reaction occurs.

FIG. 6 shows a flow chart of a gene sequencing method according to an embodiment of the present disclosure.

### DESCRIPTION OF THE PREFERRED EMBODIMENT

In order to make the objective, the technology solution and advantages of the present disclosure more clearly, the technology solution will be described more clearly and fully with reference to the accompanying drawings. It is obvious that the described embodiments are part of embodiments of the present disclosure, not all embodiments. All other embodiments obtained by those of ordinary skill in the art are within the scope of the present disclosure, based on the described embodiments of the present disclosure.

Unless otherwise defined, technical terms or scientific terms used herein should be the ordinary sense understood by those skilled in the art. “First”, “second” and similar words used in the present specification and the claims are not considered in any order, quantity or importance, but merely to distinguish between different constituent parts. Similarly, similar words such as “a” or “an” does not represent a quantity limit, but rather that there is at least one. The words “connected” or “linked” and the like are not limited to physical or mechanical connections, but may comprise electrical connections, regardless of direct or indirect. “Up”, “down”, “left”, “right” and the like are used only to represent the relative positional relationship, and when the absolute position of the object to be described changes, the relative position relation is changed accordingly.

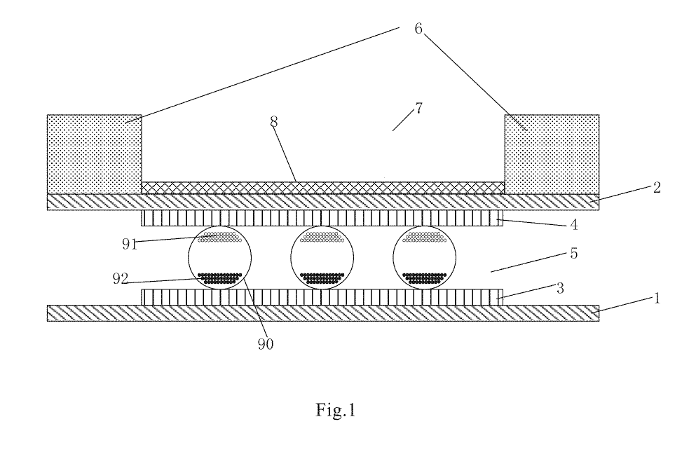

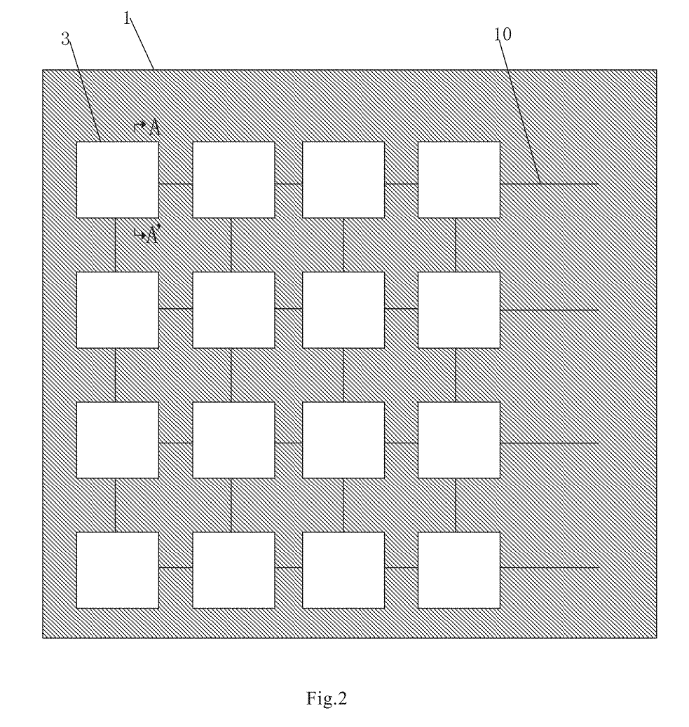

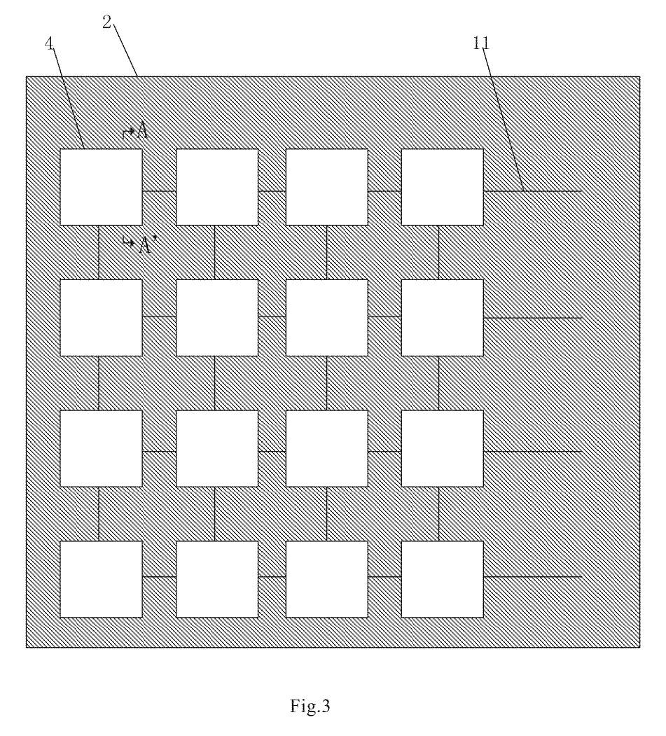

FIG. 1 shows a cross-sectional view taken along the line A-A′ in FIG. 2 and FIG. 3 of a gene sequencing chip according to the present disclosure. Referring to FIG. 1, a gene sequencing chip provided in the present disclosure invention comprises a transparent first substrate land a second substrate 2 disposed opposite to the first substrate 1. A first electrode 3 is disposed on the first substrate 1 and is a transparent electrode. A second electrode 4 is disposed on the second substrate 2. An electronic ink layer 5 is disposed between the first substrate 1 and the second substrate 2. The electronic ink layer 5 comprises a plurality of microcapsules 90 each comprising positively charged white particles 91 and negatively charged black particles 92, wherein the positively charged particles and the negatively charged particles are colored in white and black, however, the color of the two kinds of charged particles can be exchanged, and other two different colors of charged particles can be used, as long as they are easy to be distinguished. A microporous layer 6 is disposed on a side of the second substrate 2 away from the first substrate 1. Micropores 7 are formed at a position in the microporous layer corresponding to the first electrode 3. Projections of the first electrode 3, the second electrode 4 and the micropores 7 on the first substrate 1 are overlapped with each other. When a positive voltage is applied to the first electrode 3, an electric field directed from the first substrate 1 to the second substrate 2 is generated between the first substrate 1 and the second substrate 2, and a negative voltage may be applied to the second electrode 4 or two voltages may be applied simultaneously on the first electrode 3 and the second electrode 4 respectively to generate an electric field directed from the first substrate 1 to the second substrate 2 between the first substrate 1 and the second substrate 2 such that the positively charged white particles 91 and the negatively charged black particles 92 in the microcapsules 90 are distributed as shown in FIG. 1. Thus, alternatively, one of the first electrode 3 and the second electrode 4 may be removed.

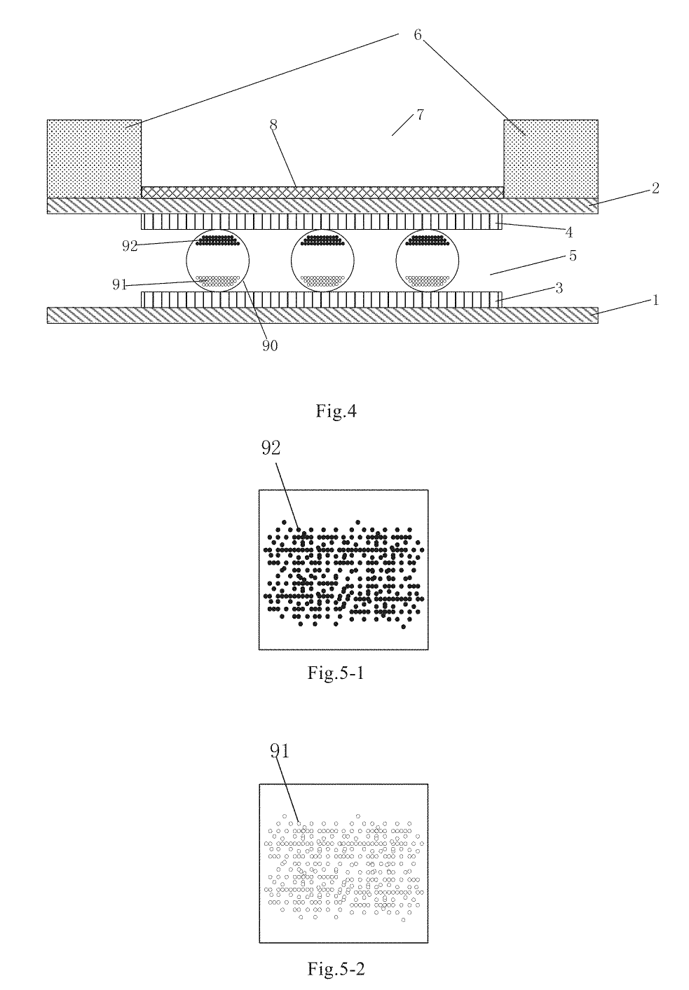

The ion-sensitive film 8 is disposed on a side of the micropores 7 close to the second substrate 2. According to an embodiment of the present disclosure, the ion-sensitive film 8 may be made of silicon nitride. Ion-sensitive film made of silicon nitride is more sensitive to hydrogen ions. When complementary basic groups pairing reaction occurs in the micropores 7, hydrogen ions are released, thereby inducing the Nernstian potential on the surface of the ion-sensitive film 8, and the electric field between the first substrate 1 and the second substrate 2 is affected to change the distribution of the charged particles in FIG. 1. Therefore the color of a part of the electronic ink layer close to the first substrate is changed and the distribution of the charged particles becomes as shown in FIG. 4. The ion-sensitive film 8 makes the color change of the electronic ink layer 5 more obvious.

FIG. 2 shows a plan view of the first substrate 1 of the gene sequencing chip of FIG. 1, and FIG. 3 shows a plan view of the second substrate 2 of the gene sequencing chip of FIG. 2. As shown in FIG. 2, the first electrode 3 may be block electrodes disposed on the first substrate 1. The block electrodes are connected with each other applied to a voltage via a first signal wire 10 by an external signal source (not shown). As shown in FIG. 3, the second electrode 4 may also be block electrodes disposed on the second substrate 2. The block electrodes are connected with each other applied to a voltage via a second signal wire 11 by an external signal source (not shown).

As shown in FIG. 1 to FIG. 3, both the first electrode 3 and the second electrode 4 are block electrodes. The first electrode 3 is disposed on a side of the first substrate 1 close to the second substrate 2, and the second electrode 4 is disposed on a side of the second substrate 2 close to the first substrate 1. It should be noted that the above description is exemplary embodiments and the shape and position of the first electrode 3 and the second electrode 4 are not limited in the present disclosure. Specifically, the first electrode 3 may be either a block electrode or a planar electrode bespreading the first substrate 1. The second electrode 4 may be either a block electrode or a planar electrode bespreading the second substrate 2. The first electrode 3 may be disposed either on a side of the first substrate 1 close to the second substrate 2 or on the other side of the first substrate 1 away from the second substrate 2. The second electrode 4 may be disposed either on a side of the second substrate 2 close to the first substrate 1 or on the other side of the second substrate 2 away from the first substrate 1. According to an embodiment of the present disclosure, the first electrode 3 may be made of indium tin oxide (ITO), the second electrode 4, the first signal wire 10, and the second signal wire 11 may be made of ITO, molybdenum, aluminum, copper or the like, and the microporous layer 6 may be made of silicon nitride or silicon oxide.

It is to be noted that the ion-sensitive film 8 is not necessary. In the case where there is none ion-sensitive film 8, the hydrogen ions generated when the complementary basic groups pairing occurs in the micropores 7 will have an impact to the electric field between the first substrate 1 and the second substrate 2, and the distribution of the charged particles is changed so that the color of a part of the electronic ink layer close to the first substrate is changed.

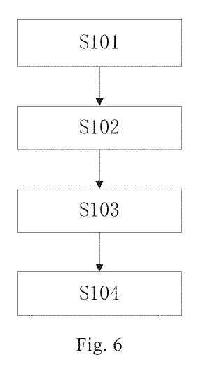

FIG. 6 is a flowchart illustrating a gene sequencing method according to an embodiment of the present disclosure.

The gene sequencing method using the gene sequencing apparatus of the present disclosure will be described below with reference to FIGS. 1, 4, 5-1, 5-2 and 6.

A gene sequencing apparatus according to an embodiment of the present disclosure may comprise the gene sequencing chip according to the present disclosure. As shown in FIG. 6, the gene sequencing method according to the gene sequencing apparatus of the present disclosure comprises the following steps:

S101: DNA microspheres containing DNA strands are added to the micropores of the gene sequencing chip for PCR amplification;

S102: a positive voltage is applied to the first electrode such that an electric field is generated between the first substrate and the second substrate, the direction of which is directed from the first substrate to the second substrate;

S103: four types of deoxyribonucleoside triphosphates are added to the micropores successively and detecting whether or not the color of a part of the electronic ink layer close to the first substrate is changed; and

S104: The type of basic group on the DNA strand is determined according to the fact that which type of the deoxyribonucleoside triphosphate is added when the color of a part of the electronic ink layer close to the first substrate is changed.

According to an embodiment of the present disclosure, the deoxyribonucleoside triphosphate used in S103 is a reversible termination of deoxyribonucleoside triphosphate comprising, for example, reversible termination of triphosphate adenine deoxyribonucleotides, reversible termination of triphosphate thymine deoxyribonucleotides, reversible termination of triphosphate cytosine deoxyribonucleotides and reversible termination of triphosphate guanine deoxyribonucleotides.

In detail, after adding the DNA microspheres containing the DNA strand to the micropores 7 for PCR amplification, a positive voltage signal is applied to the first electrode 3 via the first signal wire 10 such that an electric field is generated between the first substrate 1 and the second substrate 2, the direction of which is directed from the first substrate 1 to the second substrate 2. At the same time, the positively charged white particles 91 in the microcapsules 90 is gathered on the side close to the second substrate 2, and the negatively charged black particles 92 in the microcapsules 90 is gathered on the side close to the first substrate 1, as shown in FIG. 1 and FIG. 5-1. Since the first substrate 1 and the first electrode 3 are transparent, the color of the electronic ink layer 5 is black when viewing from a side of the first substrate 1 away from the second substrate 2.

When deoxyribonucleoside triphosphates in micropores 7 are synthesized into DNA molecules, hydrogen ions are released. Thereby an electric field, the direction of which is directed from the first substrate 1 to the second substrate 2, is generated. The electric field causes the positively charged white particles 91 in the microcapsules 90 to move towards the first substrate 1 and the negatively charged black particles 92 in the microcapsules 90 to move towards the second substrate 2. At the same time, as shown in FIGS. 4 and 54, since the first substrate 1 and the first electrode 3 are transparent, the color of the electronic ink layer 5 is white when viewing from a side of the first substrate 1 away from the second substrate 2.

If an ion-sensitive film 8 is disposed in the micropores 7, the released hydrogen ions induce Nernstian potential which will cause an electric field directed from the second substrate 2 to the first substrate 1 on the surface of the ion-sensitive film 8 causing the positively charged white particles 91 in the microcapsules 90 to move towards the first substrate 1 and the negatively charged black particles 92 in the microcapsules 90 to move towards the second substrate 2. Therefore the type of basic group on the DNA strand is determined according to the fact that which type of the deoxyribonucleoside triphosphate is added when the color of a part of the electronic ink layer 5 close to the first substrate 1 is changed.

Alternatively, in step S102, a negative voltage may be applied to the second electrode 4 or voltages may be applied simultaneously on the first electrode 3 and the second electrode 4 (for example, the voltage applied to the first electrode 3 is larger than that of the second electrode 4), such that an electric field, the direction of which is directed from the first substrate 1 to the second substrate 2, is generated between the first substrate 1 and the second substrate 2. Thus, when the deoxynucleoside triphosphate in the micropores 7 is synthesized into a DNA molecule, it has the same effect as described above which will not be described here.

As a result, when the color of a part of the electronic ink layer 5 close to the first substrate 1 is changed, if the triphosphate added in the micropores 7 is triphosphate adenine deoxyribonucleotides, the basic group on the DNA strand to be detected is adenine, if the triphosphate added in the micropores 7 is triphosphate thymine deoxyribonucleotides, the basic group on the DNA strand to be detected is thymine, if the triphosphate added in the micropores 7 is triphosphate cytosine deoxyribonucleotides, the basic group on the DNA strand to be detected is cytosine, and if the triphosphate added in the micropores 7 is triphosphate guanine deoxyribonucleotides, the basic group on the DNA strand to be detected is guanine.

After completion of the type detection of basic group of the DNA at a position, it is necessary to wash the reversible termination of deoxyribonucleoside triphosphate added into the micropores and add mercapto-reagent. Unlike ordinary deoxyribonucleoside triphosphate, the 3′-terminus of the reversible termination of deoxyribonucleoside triphosphate is connected to an azide group which can not form a phosphodiester bond during DNA synthesis and thus disrupts DNA synthesis. If the mercapto-reagent is added, the azide group breaks and forms a hydroxyl group at the original position. After the mercapto-reagent is added, the type detection of basic group at the subsequent position can be detected.

According to an embodiment of the present disclosure, an image acquisition device may be disposed on a side of the first substrate 1 away from the second substrate 2, which can be configured to capture color change of a part of the electronic ink layer 5 close to the first substrate 1.

For example, the above-described image acquisition device may be a CCD camera.

The foregoing is a preferred embodiment of the present disclosure and it should be noted that various modifications and improvement may be made by those skilled in the art without departing from the principles of the present disclosure. The scope of the present disclosure is subject to the claims.

1. A gene sequencing chip comprising:
a transparent first substrate;
a second substrate disposed opposite to the first substrate;
a first electrode which is a transparent electrode disposed on the first substrate;
an electronic ink layer disposed between the first substrate and the second substrate; and
a microporous layer disposed on a side of the second substrate away from the first substrate, wherein micropores is formed at a position in the microporous layer corresponding to the first electrode.

2. The gene sequencing chip of claim 1, wherein an ion-sensitive film is disposed on a side of the micropores close to the second substrate.

3. The gene sequencing chip of claim 2, wherein the ion-sensitive film is made of silicon nitride.

4. The gene sequencing chip of claim 1, wherein the first electrode is disposed on a side of the first substrate close to the second substrate.

5. The gene sequencing chip of claim 4, further comprising a second electrode disposed on the second substrate and disposed on a side of the second substrate close to the first substrate.

6. The gene sequencing chip of claim 5, wherein both the first electrode and the second electrode are block electrodes, and projections of first electrode and the second electrode are overlapped with each other on the first substrate.

7. The gene sequencing chip of claim 6, wherein projections of first electrode, the second electrode and the micropores are overlapped with each other on the first substrate.

8. The gene sequencing chip of claim 5, wherein the first electrode is a planar electrode bespreading the first substrate, and the second electrode is a block electrode.

9. The gene sequencing chip of claim 5, wherein the second electrode is a planar electrode bespreading the second substrate, and the first electrode is a block electrode.

10. The gene sequencing chip of claim 1, wherein the electronic ink layer comprises a plurality of microcapsules, each of which comprises positively charged particles and negatively charged particles with two difference colors respectively.

11. The gene sequencing chip of claim 5, wherein a first signal wire transmitting a voltage to the first electrode is disposed on the first substrate, and a second signal wire transmitting a voltage to the second electrode is disposed on the second substrate.

12. A gene sequencing chip comprising:
a transparent first substrate;
a second substrate disposed opposite to the first substrate;
a second electrode disposed on the second substrate;
an electronic ink layer disposed between the first substrate and the second substrate; and
a microporous layer disposed on a side of the second substrate away from the first substrate, wherein micropores is formed at a position in the microporous layer corresponding to the second electrode.

13. A gene sequencing apparatus, comprising the gene sequencing chip of claim 1.

14. The gene sequencing apparatus of claim 13, further comprises an image acquisition device which is disposed on a side of the first substrate away from the second substrate and is configured to capture the color change of a part of the electronic ink layer close to the first substrate.

15. A gene sequencing apparatus, comprising the gene sequencing chip of claim 12.

16. A gene sequencing method performed using the gene sequencing chip of claim 1 comprising the following steps:
DNA microspheres containing DNA strands are added to the micropores of the gene sequencing chip for PCR amplification;
a voltage is applied to the first electrode such that an electric field is formed between the first substrate and the second substrate, the direction of which is directed from the first substrate to the second substrate;
four types of deoxyribonucleoside triphosphates are added to the micropores successively and detecting whether or not the color of a part of the electronic ink layer close to the first substrate is changed; and
the type of basic group on the DNA strand is determined according to the fact that which type of the deoxyribonucleoside triphosphate is added when the color of a part of the electronic ink layer close to the first substrate is changed.

17. The gene sequencing method of claim 16, wherein the deoxyribonucleoside triphosphate is a reversible termination of deoxyribonucleoside triphosphate, and the gene sequencing method further comprises:
washing the reversible termination of deoxyribonucleoside triphosphate added into the micropores and adding mercapto-reagent.

18. The gene sequencing method of claim 16, wherein an image acquisition device is disposed on a side of the first substrate away from the second substrate and is configured to capture the color change of a part of the electronic ink layer close to the first substrate.

19. A gene sequencing method performed using the gene sequencing chip of claim 5 comprising the following steps:
DNA microspheres containing DNA strands are added to the micropores of the gene sequencing chip for PCR amplification;
a voltage is applied to the first electrode and the second electrode such that an electric field is formed between the first substrate and the second substrate, the direction of which is directed from the first substrate to the second substrate;
four types of deoxyribonucleoside triphosphates are added to the micropores successively and detecting whether or not the color of a part of the electronic ink layer close to the first substrate is changed; and
the type of basic group on the DNA strand is determined according to the fact that which type of the deoxyribonucleoside triphosphate is added when the color of a part of the electronic ink layer close to the first substrate is changed.

20. A gene sequencing method performed using the gene sequencing chip of claim 12 comprising the following steps:
DNA microspheres containing DNA strands are added to the micropores of the gene sequencing chip for PCR amplification;
a voltage is applied to the second electrode such that an electric field is formed between the first substrate and the second substrate, the direction of which is directed from the first substrate to the second substrate;
four types of deoxyribonucleoside triphosphates are added to the micropores successively and detecting whether or not the color of a part of the electronic ink layer close to the first substrate is changed; and
the type of basic group on the DNA strand is determined according to the fact that which type of the deoxyribonucleoside triphosphate is added when the color of a part of the electronic ink layer close to the first substrate is changed.
